(19) United States
(12) Patent Application Publication
(10) Pub. No.: US 2023/0323308 A1
(43) Pub. Date: Oct. 12, 2023

(54) IN VITRO MODEL OF LIVER STEATOHEPATITIS

(71) Applicant: GENFIT, LOOS (FR)

(72) Inventors: GUILLAUME VIDAL, SAINT-ANDRE-LEZ-LILLE (FR); ROBERT WALCZAK, LILLE (FR)

(21) Appl. No.: 18/012,670

(22) PCT Filed: Jun. 23, 2021
(86) PCT No.: PCT/EP2021/067221

§ 371 Date: Dec. 23, 2022

(30) Foreign Application Priority Data
Jun. 24, 2020 (EP) 20305699.9
Nov. 19, 2020 (EP) 20208781.3

(51) Int. Cl. C12N 5/071

(52) U.S. Cl. CPC C12N 5/0671; C12N 2501/12; C12N 2501/33; C12N 2502/081; C12N 2502/1157; C12N 2502/14; C12N 2502/28

(57) ABSTRACT

The present invention relates to methods for preparing in vitro models of nonalcoholic steatohepatitis.

The present invention relates to methods for preparing in vitro models of nonalcoholic steatohepatitis.

### BACKGROUND OF THE INVENTION

Nonalcoholic fatty liver disease (NAFLD) is a rapidly emerging public health crisis, affecting up to ⅓ of the U.S. population, 75% of type 2 diabetics, and 95% of obese individuals. Early in the NAFLD disease spectrum, non-alcoholic fatty liver (NAFL) is benign and asymptomatic, characterized by accumulation of fat within the liver's hepatocytes; however, this phenotype can progress to non-alcoholic steatohepatitis (NASH), a serious condition that includes steatosis, inflammation, and hepatocyte ballooning.

Left unaddressed, NASH may progress further to cirrhosis and or hepatocellular carcinoma, often resulting in liver transplant or death. NASH is a complex disease whose genesis is linked to a number of factors including genetics, metabolic syndrome, and/or external factors such as diet and exercise, making identification of new therapies challenging (Dongiovanni P, et al., Curr Pharm Des. 2018).

The mechanisms of action of NASH therapies under development are extremely varied and target very different aspects and stages of the disease process, making the development of a model faithfully reproducing NASH very challenging.

Though immortalized human cell lines, such as Huh7, HepG2 or iPSC-derived hepatocytes, can be induced towards a steatotic phenotype in culture, multiple studies have shown that these cells barely reflect the native liver metabolic function.

For cocultures approach, the use of poorly differentiated or functional cell lines, the lack of control of the amount and cellular ratio of the different hepatic cell types constituting the liver (Hurrell T, et al., Cells. 2020) do not fully recapitulate the NASH phenotype (InSphero liver microtissue, Mukherjee S, et al., Am J Transl Res. 2019).

To adequately mimic the complex pathology of this disease, in vivo conditions need to be carefully selected and translated into in vitro setups. Therefore, there is still an unmet need of providing an in vitro model of NASH that mimics the physiological behavior of the liver occurring during this disease.

### SUMMARY OF THE INVENTION

The invention relates to in vitro spheroid setup containing multiple human hepatic cell types, being seeded in a precise and controlled amount.

Accordingly, the invention relates to a liver spheroid adequately mimicking NASH phenotype, and methods of preparing the same.

In a first aspect, the invention relates to a method of preparing a liver spheroid comprising seeding and culturing together between 10 and 60 parts of human hepatocytes (HH), between 4 and 30 parts of human stellate cells (HSC), between 1 and 10 parts of human liver endothelial cells (LEC) and between 1 and 10 parts of Kupffer cells (KC), and culturing the cells under conditions allowing the formation of a liver spheroid. In a particular embodiment, the method comprises seeding and culturing together between 500 and 3000 HH, between 200 and 1500 HSC, between 50 and 500 LEC and between 50 and 500 KC.

Preferably, the invention relates to a method of preparing a liver spheroid comprising seeding and culturing together between 20 and 50 parts of HH, between 5 and 20 parts of HSC, between 3 and 7 parts of human LEC and between 3 and 7 parts of KC.

In another aspect, the invention relates to a method of inducing an in vitro model of NASH, wherein said method comprises the culture of the liver spheroid described herein under conditions suitable to induce a NASH phenotype.

The invention further relates to methods of use of the in vitro model of NASH of the invention.

The present invention is a relevant tool for in vitro modeling of NASH, mimicking NASH pathogenesis from steatosis to more severe disease states such as fibrosing NASH.

### BRIEF DESCRIPTION OF THE FIGURES

FIG. 1: HES (hematoxylin-eosin-saffron) coloration of spheroid after 6 days of culture in “NASH induction medium”, displaying typical NASH features: steatosis (arrows) and ballooned hepatocytes (circles)

FIG. 2: PS (Picro Sirius Red) staining of spheroid after 6 days of culture in “NASH induction medium” displaying moderate fibrosis (4.09%), indicated by red-stained collagen fibers (stars)

FIG. 3: HES coloration of InSphero 3D InSight™ liver model after 6 days of culture in “NASH induction medium”: no steatosis and many ballooned hepatocytes are visible (circles)

FIG. 4: PS staining of InSphero 3D InSight™ liver model after 6 days of culture in “NASH induction medium” displaying very high amount of fibrosis (18.88%), indicated by red-stained collagen fibers (stars)

FIG. 5: Morphology of spheroids maintained in culture for 24 h (A) and 9 Days (B) after preparation. U and S indicated ‘untreated’ and ‘stimulated’ spheroids, respectively. The change in morphology after 9 days of stimulation (S) is notable. A, B, C and D correspond to human hepatocytes donors mentioned in Table 1 and number #1 and #2 correspond to the HSC donors mentioned in Table 2, respectively. For all HH and HSC combinations, Kupffer cells and liver endothelial cells donor remained unchanged.

FIG. 6: Steatosis baseline level (expressed as Adipored RFU, relative fluorescence unit) in culture for 24 h (A) and 9 Days (B) after preparation. A, B, C and D correspond to human hepatocytes donors mentioned in Table 1 and number #1 and 2 correspond to the HSC donor mentioned in Table 2, respectively. For all HH and HSC combinations, Kupffer cells and liver endothelial cells donor remained unchanged (n=6).

FIG. 7: Steatosis level after 9 Days of stimulation. A, B, C and D correspond to human hepatocytes donors mentioned in Table 1 and number #1 and #2 correspond to the HSC donors mentioned in Table 2, respectively. For all HH and HSC combinations, Kupffer cells and liver endothelial cells donor remained unchanged (n=6).

FIG. 8: Interleukin-8 concentration at Day 0 (A) and Day 9 (B) of culture. IL-8 concentration is expressed in pg/mL. A, B, C and D correspond to human hepatocytes donors mentioned in Table 1 and number #1 and #2 correspond to the HSC donors mentioned in Table 2, respectively. For all HH and HSC combinations, Kupffer cells and liver endothelial cells donor remained unchanged (n=6).

FIG. 9: Interleukin-8 concentration after 9 days of NASH culture conditions. IL-8 concentration is expressed in pg/mL normalized over untreated (concentration of simulated sample—minus concentration of unstimulated sample; n=6). A, B, C and D correspond to human hepatocytes donors mentioned in Table 1 and number #1 and #2 correspond to the HSC donors mentioned in Table 2, respectively. For all HH and HSC combinations, Kupffer cells and liver endothelial cells donor remained unchanged.

### DETAILED DESCRIPTION OF THE INVENTION

Despite the high interest in NASH, there continues to be a lack of common therapeutic strategy, as evidenced by the over 30 different targets currently in development (PPARs modulators, anti-fibrotic/anti-inflammatory modulators, glucose pathway modulators, lipid modulators). This likely stems from a lack of disease understanding and experimental models for identifying and validating targets.

The present invention describes the reproducible and controlled formation of liver spheroids for modeling NASH.

### Definitions

As used herein, the term “about” used with respect to a number of days of cell culture represents the period of time corresponding exactly to said number of days, for example 24 hours for one day, 48 hours for 2 days, etc. but also a period with a tolerance of +/−4 hours. For example, about 1 day means 20 to 28 hours, about 2 days means 44 to 52 hours, about 6 days means 140 to 148 hours, about 7 days means 164 to 172 hours, etc.

Furthermore, the term “about” used with respect to a concentration value means +/−10% of this concentration value, such as +/−5% of this concentration value.

In the context of the present invention, the term “part” used with respect to cell types refers to the proportion of said cell types. For example, the expression “10 parts of cell A and 1 part of cell B” means that 10-times more cell A are used than cell B. This information is independent from the culture volume of the cells. As such, still with the example of “10 parts of cell A and 1 part of cell B”, this expression can mean:
10 cells A and 1 cell B;
200 cell A and 20 cell B;
1000 cell A and 100 cell B;

### Method of Preparing a Liver Spheroid

In a first aspect, the invention relates to a method of preparing a liver spheroid comprising seeding and culturing together between 10 and 60 parts of HH, between 4 and 30 parts of HSC, between 1 and 10 parts of human LEC and between 1 and 10 parts of KC, and culturing the cells under conditions allowing the formation of a liver spheroid.

### Cells

The method of preparing a liver spheroid according to the invention implements the culture of four different cell types, namely HH, HSC, human LEC and human KC.

In a particular embodiment, the cells are primary cells. In yet another embodiment, the cells are human primary cells. In a further particular embodiment, the liver spheroid is prepared from primary HH, primary HSCs, primary human LEC and primary human KC.

In a further embodiment, each cell type originates from a single donor. In the context of the present invention, the expression “each cell type originates from a single donor” means that for a given cell type, said cell type was obtained from a single donor, such as a single human, and is not a pool of cells originating from different donors, such as a pool of HH originating from different donors. Still in the context of the present invention, two or more cell types may originate or not from a single donor. For example, each of the four cell types implemented herein may originate from four different donors. In another variant, two of the four cell types may originate from a first single donor, and the two other cell types may be derived from a second single donor, or from a second donor and a third donor.

In yet another embodiment, the different cell types used in the method of the present invention are obtained from a commercial source.

The cells implemented in the present invention may be derived from donors of different backgrounds, such as a background correlated or not correlated to NAFLD or NASH progression towards more severe phenotypes. It has been shown that the genetic background and metabolic disturbance can play a pivotal role in NAFLD and NASH progression. For example, particular genetic polymorphisms portray an advanced propensity to develop NAFLD, such as the Single Nucleotide Polymorphism (SNP) patatin-like phospholipase domain-containing 3 (PNPLA3) rs738409 variant which correlates not only with the progression from simple steatosis towards NASH, but also with the severity of steatosis and fibrosis in these patients. (Romeo S, et al, Nat Genet. 2008 40(12): 1461-1465). Transmembrane 6 superfamily 2 (TM6SF2) rs58542926 probably also relate to the disease progression. The TM6SF2 gene is a master regulator of the metabolic syndrome. Decreased VLDL (Very Low Density Lipoproteins) export has been reported in NASH patients carrying the SNP TM6SF2 rs58542926 mutation, promoting steatosis and decreasing plaque formation by lowering triglyceride export into the blood stream (Kozlitina J, et al., Nat Genet. 2014, 46(4): 352-356). Accordingly, in a particular embodiment, the cells implemented in the present invention, in particular the HSC, are derived from donors having a genetic background not correlated to NAFLD or NASH disease progression. In another embodiment, the cells implemented in the present invention, in particular the HSC and/or HH, are derived from donors having a genetic background correlated to NAFLD or NASH disease progression, such as from a donor having the PNPLA3 rs738409 SNP or the TM6SF2 rs58542926 SNP. In another embodiment, cells implemented in the present invention may be derived from a donor having at least one metabolic disturbances, such as type 2 diabetes.

### Co-Culture of the Cells

The cells used in the method of the present invention are cultured in conditions suitable to prepare a liver spheroid.

According to the present invention, the four cell types described herein are seeded together in the same culture medium to form the liver spheroid, in defined amounts. More specifically, the method of the invention comprises seeding together between 10 and 60 parts of HH, between 4 and 30 parts of HSC, between 1 and 10 parts of LEC and between 1 and 10 parts of KC. Preferably, the invention relates to a method of preparing a liver spheroid comprising seeding and culturing together between 20 and 50 parts of HH, between 5 and 20 parts of HSC, between 3 and 7 parts of human LEC and between 3 and 7 parts of KC, and culturing the cells under conditions allowing the formation of a liver spheroid.

In a particular embodiment, the method comprises seeding and culturing together between 500 and 3000 HH, between 200 and 1500 HSC, between 50 and 500 human LEC and between 50 and 500 KC. In another particular embodiment, the method comprises seeding and culturing together between 1000 and 2500 HH, between 250 and 1000 HSC, between 150 and 350 LEC and between 150 and 350 KC. In a further particular embodiment, the method comprises seeding and culturing together between 1500 and 2500 HH, between 700 and 1100 HSC, between 100 and 200 LEC and between 100 and 200 KC. In yet another embodiment, the method comprises seeding and culturing together between 1800 and 2200 HH, between 810 and 990 HSC, between 135 and 165 LEC and between 135 and 165 KC. In another embodiment, the method comprises seeding and culturing together between 1900 and 2100 HH, between 850 and 950 HSC, between 140 and 160 LEC and between 140 and 160d KC. In a further particular embodiment, the method comprises seeding and culturing together 2000 HH, 900 HSC, 150 LEC and 150 KC. In particular variants of this embodiment, the cells are cultured in between 50 μL and 500 μL of culture medium, in particular between 75 μL and 200 μL, such as in about 100 μL of culture medium.

The cells are cultured in suitable plates, such as in 96 well plates, in particular plates with ultra-low attachment surfaces. In a particular embodiment, the plates have around clear bottom. Such plates are readily available from a number of manufacturers, such as from Corning.

The cells are cultured in a cell culture medium suitable to form a liver spheroid. The cell culture medium can be derived from cell culture media well known in the art (also referred to herein as a “base culture medium”), optionally supplemented with suitable components. For example, the base culture medium can be a William's E cell culture medium or Dulbecco's Modified Eagle Medium (DMEM), or advanced DMEM or any other cell culture medium suitable for the culture of hepatocytes and hepatic non-parenchymal cells. The base culture medium can also be a mixture of known culture media. The base culture medium can be supplemented with common components useful in cell culture, in particular liver cell culture, such as antibiotics, buffers, nutrients and growth factors (such as epidermal growth factor (EGF) and/or hepatocyte growth factor (HGF), preferably both). Useful supplements include, without limitation, penicillin, streptomycin, insulin, transferrin, selenium, glutamine, HEPES, gentamicin.

In a particular embodiment, insulin may be used at a concentration comprised between 0.05 μM and 4 μM, in particular between 1 and 3 μM, such as about 2 μM.

In a particular embodiment, transferrin may be used at a concentration comprised between 1 to 15 μg/mL, such as about 5 μg/mL.

In a particular embodiment, EGF may be used at a concentration comprised between 1 and 10 ng/mL, in particular between 3 and 4 ng/mL, such as about 3.33 ng/mL.

In a particular embodiment, HGF may be used at a concentration comprised between 0.1 ng/mL and 10 ng/mL, such as between 1.5 and 8 ng/mL.

In an illustrative embodiment, cell culture is implemented in a two-step process. In a first step of this embodiment, the cells are seeded in a first serum-containing culture medium and are then in a serum-free medium. In a particular embodiment, the first medium comprises serum at a concentration comprised between 1 and 10%, such as between 5 and 8%, the serum concentration being more particularly of about 7%. In a particular embodiment, both the first and second culture medium contain HGF, at a concentration comprised between 0.5 ng/mL and 10 ng/mL, such as between 1 and 7.5 ng/mL. In a particular embodiment, the second medium comprises HGF at a concentration higher than the concentration of HGF in the first medium. In yet another embodiment, the concentration of HGF in the second medium is 1.5-, 2-, 3- or 4-fold higher than the concentration of HGF in the first medium. In a particular embodiment, the concentration of HGF in the second medium is about 3-fold higher than the concentration of HGF in the first medium. In yet another embodiment, HGF in the first medium is at a concentration comprised between 0.5 ng/mL and 2.5 ng/mL, such as between 1 and 2 ng/mL, HGF being more particularly at a concentration of about 1.67 ng/mL. In a further particular embodiment, the concentration of HGF in the second medium is about 3-fold higher than the concentration of HGF in the first medium. In yet another embodiment, HGF in the second medium is at a concentration comprised between 0.75 ng/mL and 10 ng/mL, such as between 1.5 and 8 ng/mL, more particularly between 4 and 6 ng/mL, HGF being more particularly at a concentration of about 5 ng/mL.

In a further particular embodiment, the base culture medium for both the first and second culture media is identical or different. In a particular embodiment, the base culture medium is the same for both the first and second culture media. In a particular embodiment, the base culture medium comprises a mixture of DMEM and Williams media. In yet another particular embodiment, the base culture medium comprises a mixture of advanced DMEM, DMEM and Williams media. In a further particular embodiment, the base culture medium comprises ⅔ of advanced DMEM medium and ⅓ of DMEM:Williams 1:1.

In a particular embodiment, the first serum-containing culture medium and the second serum-free medium comprise insulin.

In a further particular embodiment, insulin in the first and second culture medium is at a concentration of about 1 to 3 μM, such as about 2 μM.

In a particular embodiment, the first serum-containing culture medium and the second serum-free medium comprise EGF.

In a further particular embodiment, EGF in the first and second culture medium is at a concentration of about 3 to 4 ng/mL, such as about 3.33 ng/mL.

In a particular embodiment, the first serum-containing culture medium and the second serum-free medium comprise HGF. In this embodiment, HGF may be comprised in said media in the concentration defined above.

In a particular embodiment, the first medium comprises 7% serum, penicillin/streptomycin 100 U/mL/100 μg/mL, insulin 2 μM, glutamine 2 mM, HEPES 5 mM, transferrin 5 μg/mL, bovine serum albumin (BSA) 0.27 mg/mL (such as BSA introduced into the culture medium by addition of a lipid-rich BSA formulation such as AlbuMAX; Gibco), sodium selenite 3.33 ng/mL, EGF 3.33 ng/mL and HGF 1.67 ng/mL. In a second step of this embodiment, the cells are then cultured in a serum-free medium containing penicillin/streptomycin 100 U/mL/100 μg/mL, insulin 2 μM, GlutaMax 2 mM, HEPES 5 mM, transferrin 6.25 μg/mL, selenous acid 5.26 ng/mL, BSA 1.25 mg/mL, linoleic acid 5.35 μg/mL, EGF 3.33 ng/mL, HEPES 15 mM and HGF 5 ng/mL.

The first culture step may be carried out for at least 5 days, such as for at least 6, or at least 7 days. In a particular embodiment, the first culture step is carried out for about 7 days. The second culture step may be carried out for at least 4 days, such as 5, 6, 7, 8, or 9 days. In a particular embodiment, the second culture step may be carried out for 9 days.

In a particular aspect, the invention relates to a liver spheroid obtainable according to the method described above.

### Method of Inducing a NASH-Like Phenotype in a Liver Spheroid

Another aspect of the invention relates to a method of inducing a NASH-like phenotype in a liver spheroid according to the invention.

The method of inducing a NASH-like phenotype comprises culturing the liver spheroid according to the invention under conditions suitable to induce said NASH-like phenotype. In a particular embodiment, the liver spheroid of the invention is cultured in a cell culture medium comprising components useful in the induction of the NASH-like phenotype. For example, the liver spheroid may be cultured in a NASH induction medium, in particular a NASH induction medium devoid of serum. The NASH induction medium may be selected from commercially available medium, such as the NASH induction medium sold by InSphero (ref: ID CS-07-212-01). In another embodiment, the NASH induction medium comprises: insulin at concentration comprised between 100 nM and 2 μM, free fatty acid (FFA) mix of at least 2 FFA (1 saturated and 1 unsaturated) in a ratio saturated/unsaturated comprised between 0.5 and 2 and at a final concentration between 300 μM and 1 mM, at least 2 sugars at a final concentrations comprised between 10 mM to 50 mM and cholesterol at a concentration comprised between 5 μg/mL to 500 μg/mL. Alternatively, a composition comprising:
at least one carbohydrate;
at least one free fatty acid;
insulin; and
a cholesterol source;
   can be used.

In a particular embodiment, the at least one carbohydrate corresponds to a mixture of glucose and fructose. In yet another particular embodiment, the medium comprises glucose at a concentration comprised between 0.2 g/L and 20 g/L, in particular between 0.5 g/L and 10 g/L, such as at a concentration of about 2 g/L. In a further particular embodiment, the medium comprises fructose at a concentration comprised between 0.3 g/L and 30 g/L, in particular between 1 g/L and 10 g/L, such as at a concentration of about 3 g/L.

In another particular embodiment, the at least one free fatty acid corresponds to a mixture of fatty acids, such as a mixture of one unsaturated fatty acid and one saturated fatty acid. In a particular embodiment, the at least one free fatty acid corresponds to a mixture of oleate and palmitate. In yet another particular embodiment, oleate is comprised in the medium at a concentration between 50 μM and 1 mM, in particular between 100 μM and 500 μM, in particular between 200 and 300 μM, such as about 265 μM; and palmitate is comprised in the medium at a concentration between 25 μM and 500 μM, in particular between 50 μM and 250 μM, in particular between 100 μM and 200 μM, such as 135 μM.

In a particular embodiment, insulin is at a concentration comprised between 10 nM and 10 μM, in particular between 50 nM and 500 nM, such as about 100 nM.

In another particular embodiment, the cholesterol source is cholesterol. In a further particular embodiment, cholesterol is at a concentration comprised between 5 and 500 μg/mL, in particular between 10 and 100 μg/mL, such as at about 50 μg/mL.

In yet another particular embodiment, base Williams/DMEM 50%/50% comprising insulin 100 nM, a mixture of oleate/palmitate 265 μM/135 μM, glucose 2 g/L, fructose 3 g/L and cholesterol 50 μg/mL can be used.

According to a particular aspect, the invention relates to a medium comprising:
glucose at a concentration comprised between 0.2 g/L and 20 g/L, in particular between 0.5 g/L and 10 g/L, such as at a concentration of about 2 g/L;
fructose at a concentration comprised between 0.3 g/L and 30 g/L, in particular between 1 g/L and 10 g/L, such as at a concentration of about 3 g/L;
oleate at a concentration between 50 μM and 1 mM, in particular between 100 μM and 500 μM, in particular between 200 and 300 μM, such as about 265 μM;
palmitate at a concentration between 25 μM and 500 μM, in particular between 50 μM and 250 μM, in particular between 100 μM and 200 μM, such as 135 μM;
insulin at a concentration comprised between 10 nM and 10 μM, in particular between 50 nM and 500 nM, such as about 100 nM; and
cholesterol at a concentration comprised between 5 and 500 μg/mL, in particular between 10 and 100 μg/mL, such as at about 50 μg/mL.

In a particular embodiment, the medium of the invention comprises:
insulin 100 nM;
oleate 265 μM;
palmitate 135 μM;
glucose 2 g/L;
fructose 3 g/L; and
cholesterol 50 μg/mL.

The liver spheroid is cultured in the NASH induction medium for a duration sufficient to induce the NASH-like phenotype of interest, such as a phenotype including steatosis, ballooning and inflammation, but without fibrosis (NASH-like phenotype), or further including fibrosis (fibrosing NASH-like phenotype). In a particular embodiment, the liver spheroid is cultured in the NASH induction medium for at least 4 days, such for at least 5 or at least 6 days. In another embodiment, the liver spheroid is cultured in the NASH induction for more than 6 days, such as for at least 7, 8 or 9 days. Medium change may be carried out on a regular basis, such as after about 24, 48 or 72 hours after the last medium addition or change.

Advantageously, the present invention is suitable to produce a NASH-like phenotype from a liver spheroid, correlating simple steatosis to moderate fibrosing NASH. This is particularly advantageous as compared to other liver spheroids such as the 3D InSight™ liver model marketed by InSphero, which reproduced only a severe cirrhotic-like phenotype, which is improper for the study of NASH etiology or progression and for the screening of molecules having potentially a therapeutic effect on NASH or fibrosing-NASH.

The invention also relates to a liver spheroid having a NASH-like phenotype, such as a fibrosing NASH-like phenotype, obtainable according to the method of induction described above. The liver spheroid having a NASH-like phenotype can be used as a model of NASH or fibrosing NASH.

### Method for Screening Therapeutic Substances

The invention also relates to a method for screening the potential anti-NASH effect (such as an anti-fibrosing NASH effect) of a test substance, comprising:
culturing the liver spheroid having a NASH-like phenotype (such as a fibrosing NASH-like phenotype) according to the invention with said test substance; and
determining the variation of at least one parameter in the liver spheroid or in the culture medium.

In a particular embodiment, the method comprises:
i. inducing a NASH-like phenotype in a liver spheroid according to the method described herein, or providing a liver spheroid having a NASH-like phenotype according to the invention;
ii. contacting the liver spheroid having a NASH-like phenotype with said test substance; and
iii. determining the variation of at least one parameter in the liver spheroid or in the culture medium following step ii.

However, it should be understood that steps i. and ii. can also be carried out at the same time, or step ii. can be implemented before step i., i.e. contacting the liver spheroid with the test substance does not necessarily occur after induction of the NASH-like phenotype.

The test substance can be of any type, such as a small molecule, a macromolecule or more complex substances such as viruses or parasites. Macromolecules include, without limitation, peptides, proteins and nucleic acids. The test substance can be part of a library of substances, for example a library of small molecules or a library of macromolecules. The method of the invention can also be used to assess the effect of combinations of substances.

The effect of the test substance can be assessed by determining the variation of at least one parameter in the liver spheroid or in the culture medium of the liver spheroid culture. The at least one parameter can be selected from markers relevant to the particular disease the model is used for. In particular, the at least one parameter can be selected from steatosis, fibrosis and inflammation markers. Illustrative steatosis markers that may be assessed include, without limitation: increased lipogenesis, desaturases, LOX activities, impaired peroxisomal polyunsaturated fatty acid (PUFA) metabolism. Illustrative fibrosis markers that may be assessed include, without limitation: collagen, elastin, glycoproteins, hyaluronan, Tissue Inhibitors of MetalloProteinases (TIMPs), Cartilage oligomeric matrix protein (COMP). Illustrative inflammation markers that may be assessed include, without limitation: Interleukin-8 (IL-8), monocytes chemoattractant protein-1 (MCP-1), tumor necrosis factor-α (TNF-α), interleukin-6 (IL-6), Interleukin 1 beta (IL-1β), Chemokine (C—C motif) ligand 5 (CCL-5). In particular, the at least one parameter may be selected from the group consisting of protein markers, RNA markers, liver spheroid size and liver spheroid integrity. Other parameters include lipid content of the liver spheroid, chemokines production, cytokines production and collagen production.

### EXAMPLES

### Example 1: Preparation of a Spheroid of the Invention

### a—Establishment of Spheroids

Stellate cells (HSC, Innoprot) and liver endothelial cells (LEC, Innoprot) were thawed, several days before spheroid formation, respectively in SteCM (Innoprot) supplemented with 1% SteCGS complements, 1% penicillin/streptomicin and 2% fetal bovine serum (Innoprot, Ref P60126) and in ECM supplemented with 1% ECGS complements, 1% penicillin/streptomicin and 5% fetal bovin serum (Innoprot, Ref P60104) for their amplification.

Primary human hepatocytes (HH from KalyCell) and Kupffer cells (KC, Lifenethealth) were thawed, respectively in UCRM (In Vitro ADMET Labatories Ref 81015) and RPMI (Corning, Ref 10-040-CVR), both supplemented with 1% penicillin/streptomicin and 10% fetal bovin serum. Cells were seeded in 96 well ULA plate (Ultra-Low Attachment surface; Corning) in 100 μL home-made aggregation medium (⅔ Advanced DMEM medium+⅓ DMEM/Williams 1:1) 7% serum and penicillin/streptomycin 100 U/mL/100 μg/mL, insulin 2 μM, GlutaMax 2 mM, HEPES 5 mM, transferrin 5 μg/mL, AlbuMAX 0.27 mg/mL, Sodium selenite 3.33 ng/mL, epidermal growth factor (EGF) 3.33 ng/mL and hepatocyte growth factor (HGF) 1.67 ng/mL.

Then, the four cell types were seeded together in the following amounts: 2000 HH, 900 HSC, 150 LEC and 150 KC. After 7 days of culture (D-1) in the home-made aggregation medium, 50% medium change was performed twice in 24 h with DMEM/Williams 1:1 medium containing penicillin/streptomycin 100 U/mL/100 μg/mL, insulin 204, GlutaMax 2 mM, HEPES 5 mM, transferrin 6.25 μg/mL, selenous acid 5.26 ng/mL, bovine serum albumin (BSA) 1.25 mg/mL, linoleic acid 5.35 μg/mL, EGF 3.33 ng/mL, HEPES 15 mM and HGF 5 ng/mL.

The obtained liver microtissue is a 3D, primary human-cell based spheroid having a controlled cellular composition for the 4 cell types cited above.

The 3D InSight™ liver model, consisting of 10 donors-pooled primary human hepatocytes in co-culture with NPC (non-parenchymal cells) and hepatic stellate cells, was purchased from InSphero (Catalogue number MT-02-302-05, production lot hLiMT_368, stellate cells lot IPHS_11, hepatocyte lot IPHH_17, NPC cell lot IPHN_14) and cultivated with 3D InSight™ Human Liver NASH Induction medium (product ID CS-07-212-01)

### b—NASH Phenotype Induction

InSphero 3D InSight™ liver model and spheroid of example 1 were maintained in NASH induction medium (ref: ID CS-07-212-01, InSphero) without FBS for 6 days, with medium renewal after 72 h.

### c—Tissue Embedding, Staining and Histological Examinations

The InSphero model and the spheroid of example 1 were kept in 70% ethanol-eosin solution and embedded in Histogel (Thermo Scientific, HG4000). Then, the spheroids were dehydrated in ethanol solutions (baths at 70% and 100% ethanol), and incubated in two baths of Isopropanol, followed by two baths in liquid paraffin (60° C.).

The spheroids were then put into racks that were gently filled with Histowax® to completely cover the tissue. The paraffin blocks containing the spheroids were removed from the racks, stored at room temperature and cut into 3 μm slices.

For Hematoxylin/Eosin/Safranin staining, the spheroids slices were deparaffinized, rehydrated and incubated for 3 minutes in Mayer's Hematoxylin (Microm, cat #F/C0303). Then, the spheroids sections were rinsed in water and incubated 1 minute in a solution containing Eosin Y 0.5% alcoholic (VWR, cat #1.02439.0500) and Erythrosin 0.5% (VWR, cat #1.15936.0010), and rinsed in with ethanol. Sections were then incubated for 2 minutes in Safranin, eventually dehydrated and mounted using the CV Mount medium (Leica, cat #046430011).

For picrosirius red and fast green stainings, the spheroids slices were deparaffinized, rehydrated and incubated for 15 minutes in 0.04% Fast Green solution (Sigma, F7258). Then, the spheroids sections were rinsed in acetic acid solution and in water. The sections were incubated 30 minutes in a Picrosirius Red 0.1% (Alfa Aesar, B21693; Sigma P6744)—Fast Green 0.04% solution, and rinsed in with ethanol. Sections were then dehydrated and mounted using the CV Mount medium (Leica, cat #046430011).

The histological examinations and scoring were performed blindly. Images were acquired using Panoramic 250 Flash II digital slide scanner (3DHistech). Each section was examined and analyzed in QuantCenter software (3DHistech).

For each microtissue stained with Hematoxylin/Eosin/Safranin, the histological lesions due to NASH (Steatosis and ballooning) were attributed according to the NASH Clinical Research Network. For each microtissue stained with picrosirius, percentage of fibrosis was assessed by morphometric quantification of picrosirius (PS) positive area relative to the spheroid section area.

### d—Results

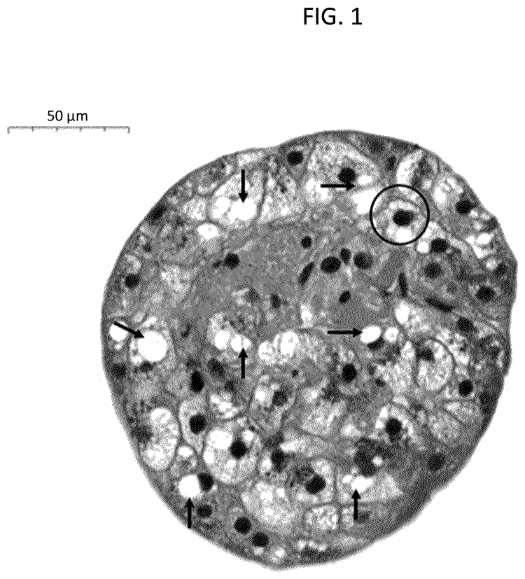

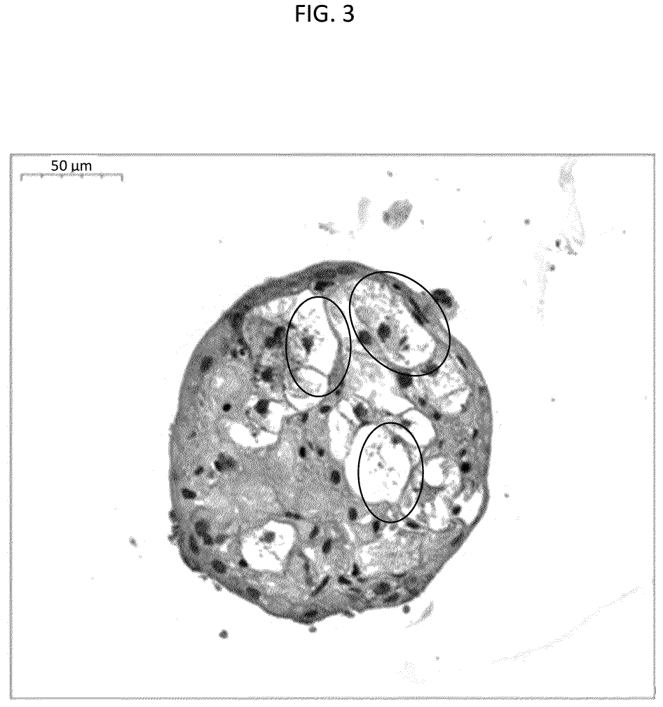

Both InSphero model and spheroid of the invention were carried out by culture in ‘NASH induction medium’ (ref: ID CS-07-212-01, InSphero) following the same protocol. The phenotypes obtained are those depicted in FIGS. 1, 2, 3 and 4.

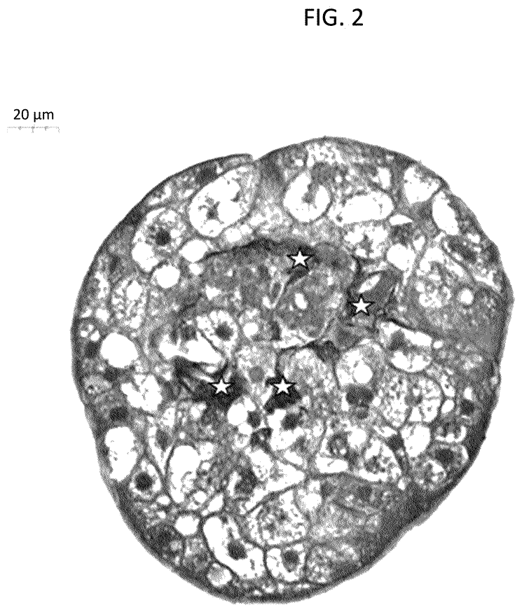

With the spheroid of the invention (FIGS. 1 and 2), key NASH features manifested by hepatic cells are present: steatosis, ballooning (FIG. 1) and collagen fibers (4.09% of fibrosis, FIG. 2).

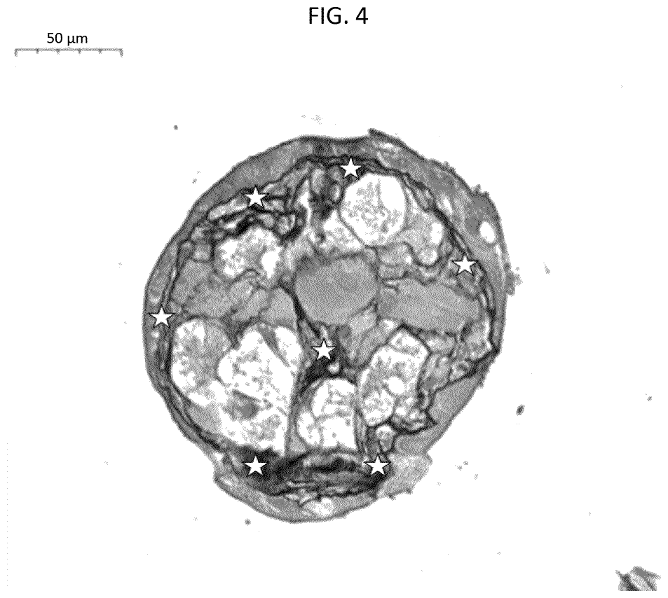

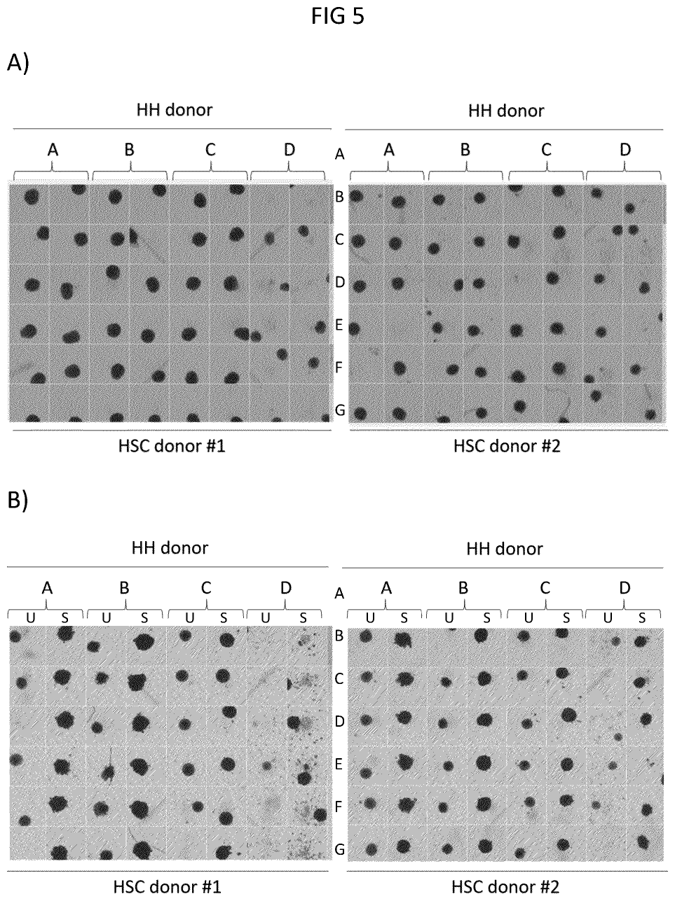

With the InSphero™ liver model, no steatosis, but many ballooned hepatocytes were observed, and the percentage of collagen fibers represented nearly 19% (18.88%) of the liver model slice surface (FIG. 4).

A parallel can be drawn about the surface occupied by collagen fibers and fibrosis scoring (Tsochatzis E, et al., J Hepatol. 2014 May; 60(5): 948-54) and it can be seen that collagen fibers form an important network everywhere in the InSphero liver model (FIG. 4).

Such a quantity of fibers observed in the InSphero™ liver model can be assimilated to a stage F3 or even F4 (cirrhosis). Cirrhosis is the common evaluation criterion for the various etiologies of liver injury and represents the most serious histological stage. Moreover, the notation of the fibrotic stages in the liver integrates a structural component: high amount of collagen fibers form bridge structure in the liver.

In the same culture conditions as the spheroid of the invention, 3D InSight™ liver model leads to a histologically characterized phenotype closer to cirrhosis (no steatosis, lot of ballooning and strong fibrosis) than NASH.

On the contrary, the spheroid of example 1 of the invention leads to a model mimicking fibrosing NASH phenotype: steatosis, few ballooned cells and moderate fibrosis. Therefore, the spheroid of the invention represent a more relevant model of fibrosing NASH.

### Example 2: Construction of a Spheroid Including Cells Harboring Specific Mutations and/or Metabolic Disturbances

### Material and Methods

TABLE 1
health profile, NAS score, BMI (Body Mass Index), PNPLA3 &
TM6SF2 genotypes of the hepatocyte donors used in example 2.

| Human | | | | |
|---|---|---|---|---|
| hepatocyte | | | | |
| donor | A | B | C | D |
| Health | No | ND | NAFLD & | NAFLD & |
| profile | NAFLD | | T2D | T2D |
| NAS | / | / | 4 | 7 |
| Fibrosis stage | / | / | F1A | F2 |
| BMI | 23.1 | 24 | 31.7 | 34.4 |
| PNPLA3 | wt | wt | I148M mutant | wt |
| genotype | | | heterozygote | |
| TM6SF2 | wt | E167K mutant | wt | wt |
| genotype | | homozygote | | |

NAFLD: nonalcoholic fatty liver disease;
T2D: type-2 diabetes;
NAS: NAFLD activity score;
BMI: body mass index

TABLE 2
PNPLA3 & TM6SF2 genotypes of the donors of the stellate
cells used in example 2.

| Human stellate cell donor | #1 | #2 |
|---|---|---|
| PNAPLA3 genotype | I148M mutant heterozygote | wt |
| TM6SF2 genotype | wt | wt |

To prepare the spheroids, the 4 cell types were seeded together according to example 1 in the following amounts: 2000 HH (donor A, B, C or D), 900 HSC (donor #1 or #2), 150 LEC and 150 KC (see example 1). After 7 days of aggregation (D-1), 50% medium change was performed twice with DMEM/Williams 1:1 medium without FBS+penicillin/streptomycin 100 U/mL/100 μg/mL, insulin 2 μM, GlutaMax 2 mM, HEPES 5 mM, transferrin 5 μg/mL, AlbuMAX 0.27 mg/mL, sodium selenite 3.33 ng/mL, EGF 3.33 ng/mL and HGF 1.67 ng/mL.

The spheroids were stimulated in a DMEM:Williams 1:1 mixture complemented with insulin 100 nM, oleate/palmitate 265 μM/135 μM, glucose 2 g/L, fructose 3 g/L and cholesterol 50 μg/mL.

### Genotyping

Cells were genotyped for PNPLA3 I148M and TM6SF2 K167E mutations. Briefly, total DNA was isolated from cells using DNeasy blood & tissue kit (Qiagen, Ref 69506). Real-time qPCR was performed on a CFX96 thermocycler (BioRad) using Taqman probes with SNP ID rs738409 for PNPLA3 and rs58542926 for TM6SF2 (Thermofisher, Ref 4351379).

### Spheroid Imaging

Spheroids images were generated with the Cell3iMager neo device (CC-3000, Screen, Jp)

### Steatosis Measurement

Lipid staining and quantification were performed with the AdipoRed™ Adipogenesis Assay Reagent (Lonza). The fluorescence intensity (Relative Fluorescence Units RFU) was quantified with plate reader (TECAN; λexc: 485 nm and λem: 572 nm).

### Interleukin-8 Measurements

Spheroids supernatants were collected at Day 0 and Day 9 and stored at −80° C. IL-8 concentrations were determined using an ELISA kit (R&D Ref DY208,). Results are expressed over untreated spheroids (without molecules cocktail to induce NASH).

### Statistical Analysis

Non parametric test (Mann-Whitney or Kruskal-Wallis) were performed with Graphpad on results over untreated samples for all tests.

### Results

### Establishment of Stable Spheroids having Mutations

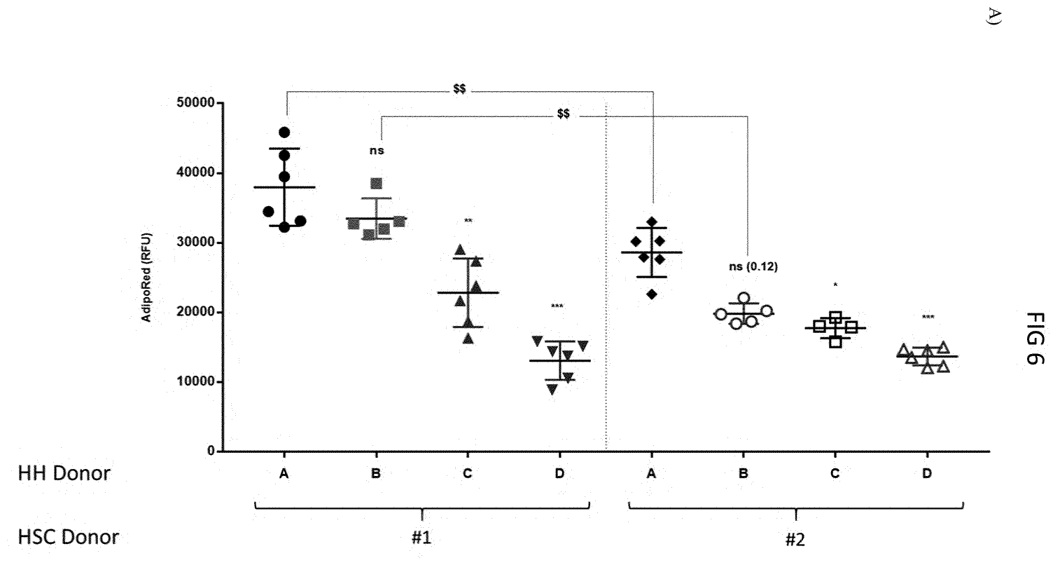

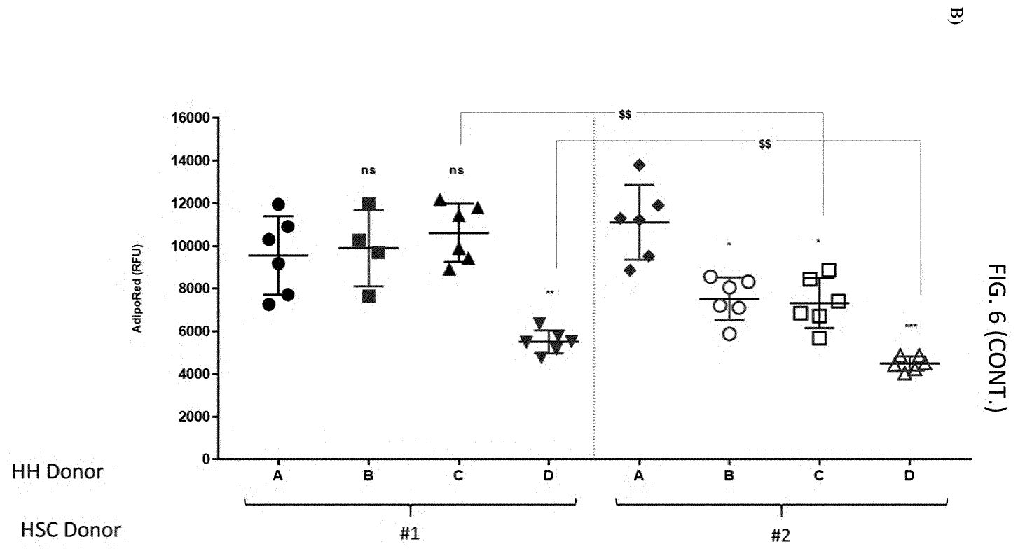

FIG. 5A shows the morphology of mutated spheroids of the invention at Day 0. The spheroids have defined composition, integrating human hepatocytes and human stellate cells with mutations and characteristics described in Table 1 and Table 2.

Also, spheroids with either HH donor A, B, C or D in combination with KC, LEC and HSC donor 1# or #2 were stable for 9 days in unstimulated culture conditions (U). They can be driven to NASH phenotype after 9 days of stimulation (S) and displayed typical altered morphology characteristic of a steatotic phenotype with irregular shape compared to unstimulated condition (U). (FIG. 5B).

### Spheroid Steatosis at Day 0

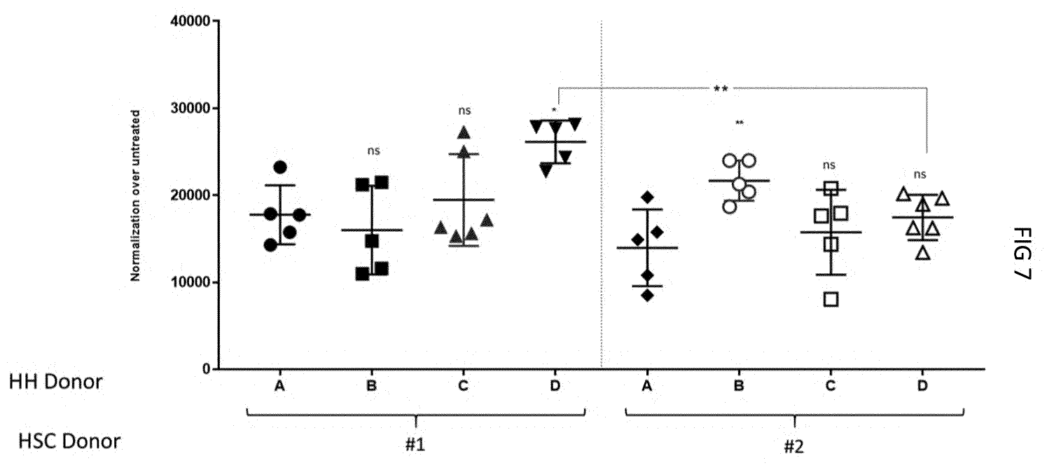

During formation, spheroid with HH donor C or D accumulated less fat than donors A or B when combined with HSC donor #1 (FIG. 6A).

Spheroid with HH donor A or B accumulated less fat when combined with HSC donor #2 than HSC donor #1 (FIG. 6A).

### Spheroid Steatosis at Day 9

When combined with HSC #1, spheroids generated from HH from donor D accumulated less fat than those generated from HH from donor A, B or C. When combined with HSC donor #2, spheroid with HH donor B, C or D accumulates less fat than spheroid with HH donor A (FIG. 6B)

This spheroid is suitable for the modeling of NASH. Moreover, it allows for the characterization of phenotypic differences due to the impact of metabolic disturbances and SNPs on spheroid steatosis.

### Steatosis Induction with NASH Culture Conditions

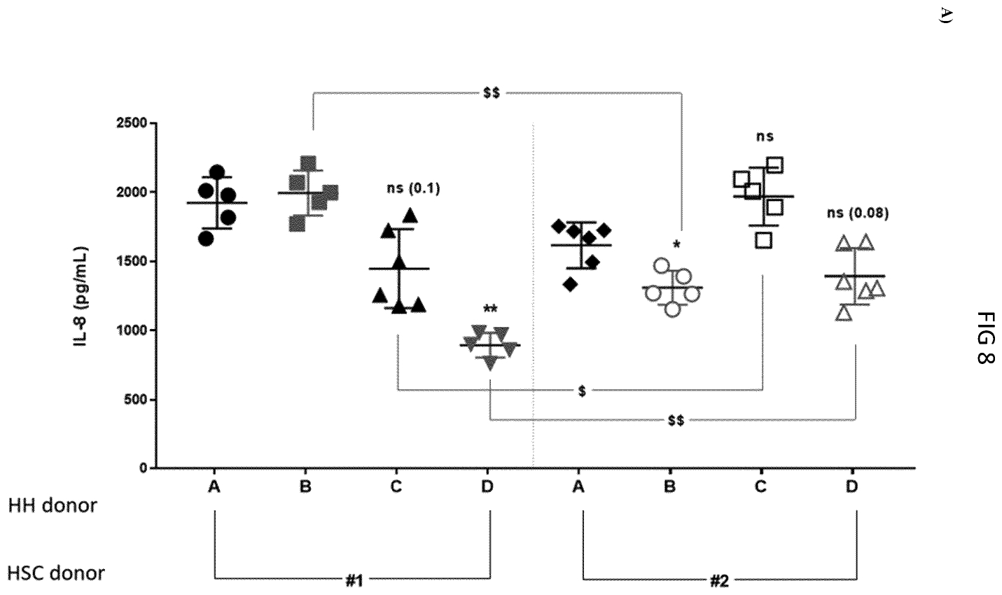

Spheroids with HH from donor D in combination with HSC from donor #1 (HSC #1) accumulated significantly more fat than spheroids with HH from donor A combined to HSC #1. Spheroids with HH from donor B combined with HSC from donor #2 (HSC #2), accumulated more fat than spheroids with HH from donor A with HSC #2. Spheroids with HH from donor D combined with HSC #1 accumulated more fat than spheroids with HH from donor D combined with HSC #2 (see FIG. 7).

Therefore, physiologically significant inter-individual variation was observed across spheroids for fat accumulation in both untreated culture conditions and in response to NASH culture conditions.

### Proinflammatory Cytokine Production

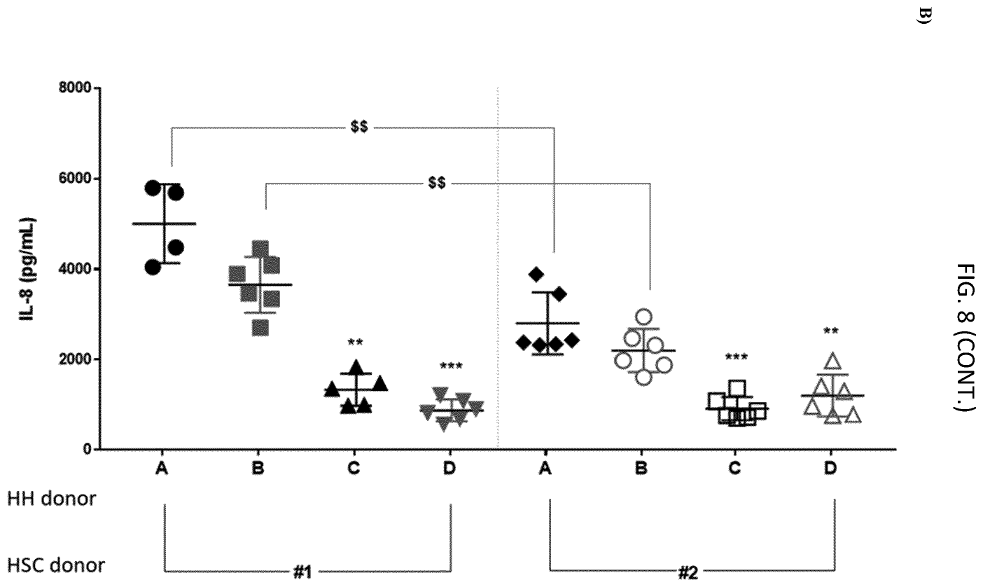

Combined with HSC #2, HH from donor C or D had higher levels of IL-8 than those combined with HSC #1 (FIG. 8A). After 9 days of culture, spheroids with HH from donor C or D when combined with both HSC #1 and HSC #2 had lower levels of IL-8 compared to spheroid with HH from donor A. Combined with HSC #2, spheroids with HH from donor A or B had lower IL-8 levels compared to spheroids with HSC #1 (FIG. 8B).

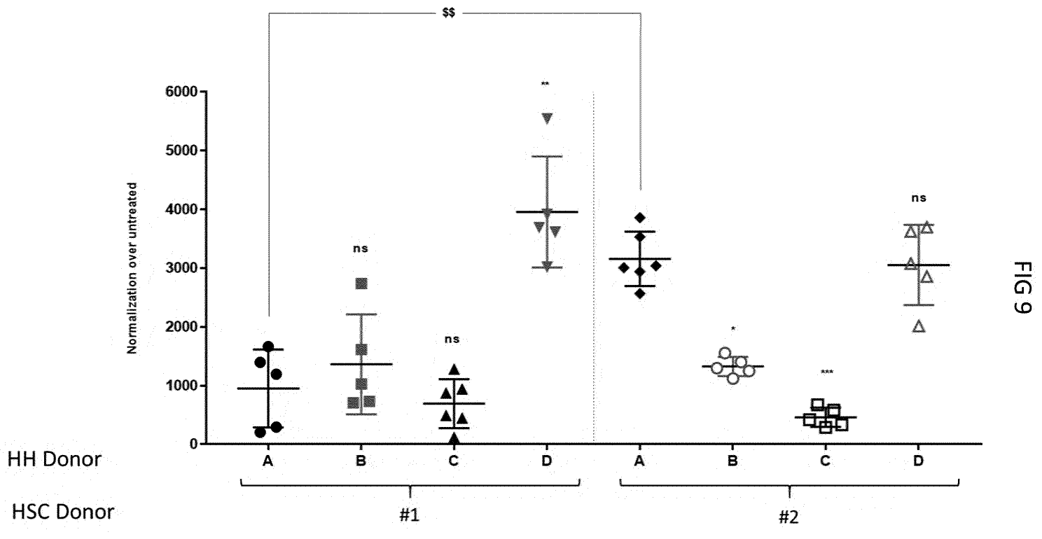

Spheroids with HH from donor D and HSC #1 produced significantly higher levels of IL-8 than spheroids with HH from donor A and HSC #1. Spheroids with HH from donor A and HSC #2 produced significantly higher levels of IL-8 than spheroids with HH from donor A and HSC #1. Spheroids with HH from donor B or C and HSC #2 produced significantly lower levels of IL-8 than spheroids with HH from donor A and HSC #2 (FIG. 9).

### Conclusion

Spheroids of the invention showed histologically confirmed steatotic, inflammatory and fibrotic phenotype induction characteristic of fibrosing NASH phenotype (example 1). Moreover, significant inter-individual variation can be observed across spheroids in their response to NASH culture conditions (example 2). Indeed, the co-culture of HHs, HKs, LEC and HSCs in the spheroids of the invention generates hepatic spheroids that mimic the observed features of NASH (steatosis, ballooning, production of pro-inflammatory cytokines). The impact of the genetic background (I148M PNPLA3 & TM6SF2 E617K mutations and metabolic disturbances) demonstrates that this NASH spheroid model is suited for investigating the genetic basis and risk factors impact on the progress of the disease. The precise amount of the 4 hepatic cell types (hepatocytes, stellate cells, kupffer cells and endothelial cells), different genetic backgrounds and inclusion of cells derived from donors with various etiologies (like Type 2 diabetus Mellitus, high BMI) can be combined to investigate the effects of these factors on disease phenotype.

The modular characteristic of this model allows the use of specific hepatic cell types with specific genetic backgrounds allowing investigation of molecular and metabolic pathways driving NASH.

1-13. (canceled)

14. A method of preparing a three dimensional (3D) liver spheroid, the method comprising seeding together between 20 and 50 parts of human hepatocytes (HH), between 5 and 20 parts of human stellate cells (HSC), between 3 and 7 parts of human liver endothelial cells (LEC) and between 3 and 7 parts of Kupffer cells (KC), and culturing the cells under conditions allowing the formation of a liver spheroid.

15. The method according to claim 14, wherein the cells are human primary cells.

16. The method according to claim 14, wherein the method comprises seeding and culturing together between 1800 and 2200 HH, between 810 and 990 HSC, between 135 and 165 LEC and between 135 and 165 KC.

17. The method according to claim 16, wherein the method comprises seeding and culturing together 2000 HH, 900 HSC, 150 LEC and 150 KC.

18. The method according to claim 14, the method comprising:
a first step of culturing the cells in a medium comprising serum and hepatocyte growth factor (HGF); and
a second step of culturing the cells in a serum-free medium comprising HGF;
wherein the concentration of HGF in the second medium is at least 1.5-fold the concentration of HGF in the first medium.

19. The method according to claim 18, wherein the first step is carried out for at least 5 days.

20. The method according to claim 18, wherein the first step is carried out for about 7 days.

21. The method according to claim 18, wherein the second step is carried out for at least 4 days.

22. A liver spheroid obtainable according to the method of claim 14.

23. A method of inducing a NASH-like phenotype in a liver spheroid, comprising the culture of the liver spheroid according to claim 22 under conditions suitable to induce said NASH-like phenotype.

24. The method according to claim 23, wherein the liver spheroid is cultured in a medium comprising:
glucose at a concentration between 0.2 g/L and 20 g/L;
fructose at a concentration between 0.3 g/L and 30 g/L;
oleate at a concentration between 50 μM and 1 mM;
palmitate at a concentration between 25 μM and 500 μM;
insulin at a concentration between 10 nM and 10 μM; and
cholesterol at a concentration between 5 and 500 μg/mL.

25. The method according to claim 24, wherein the liver spheroid is cultured in said medium for at least 4 days.

26. The method according to claim 24, wherein the liver spheroid is cultured in said medium for about 9 days.
